(19) United States
(12) Patent Application Publication
(10) Pub. No.: US 2014/0127326 A1
(43) Pub. Date: May 8, 2014

(54) Detection of Cancer by Volatile Organic Compounds From Breath

(71) Applicants: Anil K. Sood, Pearland, TX (US); Alpa M. Nick, Houston, TX (US); Edward A. Felix, Cypress, TX (US)

(72) Inventors: Anil K. Sood, Pearland, TX (US); Alpa M. Nick, Houston, TX (US); Edward A. Felix, Cypress, TX (US)

(21) Appl. No.: 14/003,155

(22) PCT Filed: Mar. 5, 2012
(86) PCT No.: PCT/US12/27778

§ 371 Date: Nov. 11, 2013

Related U.S. Application Data
(60) Provisional application No. 61/449,434, filed on Mar. 4, 2011.

(51) Int. Cl. G01N 33/497

(52) U.S. Cl. CPC G01N 33/497
USPC 424/649; 73/23.3; 73/864.51

(57) ABSTRACT

Provided are methods for detecting a cancer, such as an ovarian cancer. In certain aspects, the methods involve detecting or measuring one or more volatile organic compounds (VOCs) from the breath of a subject. Apparatuses for the collection of VOCs are provided.

This application claims priority to U.S. Application No. 61/449,434 filed on Mar. 4, 2011, the entire disclosure of which is specifically incorporated herein by reference in its entirety without disclaimer.

This invention was made with government support under T32CA101642 awarded by the National Institutes of Health. The government has certain rights in the invention.

### BACKGROUND OF THE INVENTION

1. Field of the Invention

The present invention relates generally to the fields of molecular biology and medicine. More particularly, it concerns methods for detecting cancer in a subject.

2. Description of Related Art

The paucity of information regarding a defined preclinical state indicating the presence of an ovarian cancer has resulted in an urgent need for better diagnostic modalities capable of early detection of ovarian cancer. The high mortality rate of ovarian carcinoma is attributed in part to the lack of an adequately sensitive screening modality. For example, CA 125 provides utility in assessing response to chemotherapy, detecting disease recurrence and distinguishing malignant from benign pelvic masses. However, CA 125 elevations are noted in only about 50-60% of patients with stage I disease. Thus, a void exists for diagnosis of ovarian carcinoma in its earliest stages when outcomes are significantly improved (Bast et al., 2005). Efforts to improve diagnostic methods have been made, but many of these studies suffer from the evaluation of thousands of variables across a small sample size which can result in mistakenly discriminating individuals in the sample set with predictors that are not truly predictive of the presence or absence of disease (Ransohoff, 2004). Clearly, there exists a need for new methods for detecting cancer in a subject.

### SUMMARY OF THE INVENTION

The present invention overcomes limitations in the prior art by providing new methods for detecting the presence of, susceptibility to, predisposition for, and/or risk of developing or suffering from cancer in a subject. In certain aspects, differential expression of certain volatile organic compounds (VOCs) in the breath of a subject can be used to detect the presence of a tumor or a cancer, such as an ovarian cancer, in a subject. The relative amounts of one or more volatile organic compounds in the breath of a subject may also be used to detect and/or distinguish between a cancer and a benign tumor, a pre-cancerous tumor, or a tumor of low malignant potential.

The present invention may be used, in some embodiments, to discriminate pelvic masses preoperatively as being cancerous or having an increased risk of being cancerous. In some embodiments, the present invention may be used to monitor a response to a therapy and/or to monitor for disease recurrence following completion of primary therapy. Compounds including one or more lysophosphotidic acids, prostaglandins, eicosanoids lipids and isoprostanes may be used in correlation with detection of a VOC, e.g., using SPME, to detect the susceptibility to, predisposition for, presence of, and/or risk of developing or suffering from cancer in a subject.

Also provided are methods and apparatuses for collecting a breath sample from a subject. For example, various SPME portable field breath samplers with a mouthpiece are provided and may be used, e.g., for the collection or evaluation of one or more volatile organic compound from the breath of a human subject for subsequent analysis.

An aspect of the present invention relates to a method of detecting the presence of, or an increased risk of, an ovarian or endometrial cancer in a subject, comprising detecting or measuring one or more volatile organic compound (VOC) from the breath of the subject; wherein a differential level the VOC as compared to a control indicates that the subject has, or has an increased risk of having, the cancer. The differential level may be an increased level, a decreased level, or an absence of the VOC as compared to a control. In some embodiments, said control is a control level or a reference level, although in some embodiments, the control may be a control sample. The one or more VOC may comprise at least one, two, three, four, five, six, seven or eight of 1H-imidazole-4-carboxaldehyde, nahtho[2,3-c]furan-1(3H)-one,6-hydroxy-5,7-dimethoxy, 2-ethenyl-3-ethylpyrazine, 2,2,6-trimethyl octane, {[1,4 ′]bipiperidinyl-4′-carboxamide,1-(4′ chlorobenezes)}, oxime-methoxy-phenyl, 1-hexanol-2-ethyl, or butyrolactone. In some embodiments, the one or more VOC comprises 1H-imidazole-4-carboxaldehyde and 2-ethenyl-3-ethylpyrazine. In some embodiments, the one or more VOC comprises at least two, three, four, or all of 1H-imidazole-4-carboxaldehyde, nahtho[2,3-c]furan-1(3H)-one,6-hydroxy-5,7-dimethoxy, 2-ethenyl-3-ethylpyrazine, 2,2,6-trimethyl octane, and {[1,4’]bipiperidinyl-4′-carboxamide,1-(4′ chlorobenezes)}. The one or more VOC may comprises all of 1H-imidazole-4-carboxaldehyde, nahtho[2,3-c]furan-1(3H)-one,6-hydroxy-5,7-dimethoxy, 2-ethenyl-3-ethylpyrazine, and 2,2,6-trimethyl octane. In certain aspects, an increased level of butyrolactone or {nahtho[2,3-c]furan-1(3H)-one, 6-hydroxy-5,7-dimethoxy} as compared to a control indicates that the subject has, or has an increased risk of having, the cancer. In certain aspects, a decreased level of oxime-methoxy-phenyl, 1-hexanol-2-ethyl, 1H-imidazole-4-carboxaldehyde, 2-ethenyl-3-ethylpyrazine, 2,2,6-trimethyl octane, or {[1,4′]bipiperidinyl-4′-carboxamide, 1-(4′ chlorobenezes)} as compared to a control indicates that the subject has, or has an increased risk of having, the cancer. In certain aspects, the absence of {[1,4′]bipiperidinyl-4′-carboxamide, 1-(4′ chlorobenezes)} indicates that the subject has, or has an increased risk of having, the cancer. The subject may be a mammal, such as a human. The method may comprise having the subject breathe onto a solid phase microextraction (SPME) fiber. The SPME fiber may be comprised in a portable apparatus or a point of care apparatus. The VOC may be detected from the SPME fiber via gas chromatography/mass spectroscopy (GC/MS). Said measuring may comprise detecting the VOC via gas chromatography (GC) or gas chromatography/mass spectroscopy (GC/MS). In some embodiments, the subject has the ovarian or endometrial cancer. In other embodiments, the subject does not have the ovarian or endometrial cancer. The method may further comprises administering an anti-cancer therapy to the subject.

VOC from the breath of a subject may be collected in a sample, e.g., on a filter, either directly or indirectly. For example, in some embodiments, the breath sample is directly obtained from a subject at or near the laboratory or location where the biological sample will be analyzed. In other embodiments, the breath sample may be obtained by a third party and then transferred, e.g., to a separate entity or location for analysis. In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some further aspects, the method may further comprise reporting the determination or test results to the subject, a health care payer, an attending clinician, a pharmacist, a pharmacy benefits manager, or any person that the determination or test results may be of interest.

Another aspect of the present invention relates to an apparatus comprising a mouthpiece coupled to a housing, wherein the housing comprises a solid phase microextraction fiber, wherein the apparatus is configured to capture one or more volatile organic compound (VOC) the breath of a subject on the solid phase microextraction fiber when the subject breathes into the mouthpiece. The apparatus may further comprise an apparatus configured to collect exhaled breath condensate. The solid phase microextraction fiber may contain one or more of oxime-methoxy-phenyl, 1-hexanol-2-ethyl, and butyrolactone from the breath of the subject. The solid phase microextraction fiber comprises a fiber selected from the list consisting of carboxen and polymethylsiloxane (CAR/PDMS), divinylbenzene/carboxen/polydimethylsiloxane (DVB/CAR/PDMS), polydimethylsiloxane (PDMS) metal alloy, Carbopack-Z fiber, polyacrylate (PA), Carbowax-polyethylene glycol (PEG), Carbowax/template resin (CW/TPR), and polydimethylsiloxane/divinylbenzene (PDMS/DVB). The solid phase microextraction fiber may be a carboxen and polymethylsiloxane (CAR/PDMS) solid phase microextraction fiber. The solid phase microextraction fiber may be coupled to a needle. The apparatus may further comprise a septum piercing housing needle coupled to the solid phase microextraction fiber. The mouthpiece and the housing may be unitary or modular. The apparatus may comprise a plunger, wherein the plunger is coupled to the solid phase microextraction fiber such that movement of the plunger can result in the movement of the solid phase microextraction fiber into or out from the needle. The mouthpiece may have an internal diameter of about 10 mm to about 20 mm, or about 14 mm. The housing may comprise an aperture or venting hole, wherein the aperature or venting hole allows the mammalian subject to breathe through the mouthpiece. The housing may comprise one aperture or venting hole. The aperture or venting hole may be about 2-10 mm in diameter. The housing may comprise more than one aperture or venting hole. The aperture or venting hole may be about 2-10 cm from the proximal end of the mouth piece.

As used herein, “increased level” refers to an elevated or increased amount of a compound in a sample (e.g., a VOC in a breath sample) relative to a suitable control (e.g., a non-cancerous sample or a reference standard), wherein the elevation or increase in the level of the compound in the sample is statistically-significant (p<0.05). Whether an increase in the amount of a VOC in a breath sample from a subject with a cancer relative to a control is statistically significant can be determined using an appropriate t-test (e.g., one-sample t-test, two-sample t-test, Welch's t-test) or other statistical test known to those of skill in the art.

As used herein, “decreased level” refers to a reduced or decreased amount of a compound in a sample (e.g., a VOC in a breath sample) relative to a suitable control (e.g., a non-cancerous sample or a reference standard), wherein the reduction or decrease in the level of the compound in the sample is statistically-significant (p<0.05). In some embodiments, the reduced or decreased level of gene expression can be a complete absence of a VOC in a breath sample. Whether a decrease in the amount of a VOC in a breath sample from a subject with a cancer relative to a control is statistically significant can be determined using an appropriate t-test (e.g., one-sample t-test, two-sample t-test, Welch's t-test) or other statistical test known to those of skill in the art.

Any embodiment of any of the present systems, apparatuses, devices, and methods can consist of or consist essentially of—rather than comprise/include/contain/have—any of the described elements and/or features. Thus, in any of the claims, the term “consisting of or “consisting essentially of can be substituted for any of the open-ended linking verbs recited above, in order to change the scope of a given claim from what it would otherwise be using the open-ended linking verb.

The term “coupled”, as used herein, is defined as connected, although not necessarily directly, and not necessarily mechanically.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or ” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Preclinical breath collection chamber. Chamber, SPME fiber (arrow) and holder for breath sample collection.

FIGS. 2A-B. Clinical breath collection. Patients are asked to breathe normally into a disposable mouthpiece. Breath is pre-concentrated on a PDMS and carboxen coated SPME, thermally desored with gas chromatography and identified with mass spectroscopy

FIGS. 3A-C. Comparisons of VOCs in tumor bearing versus control mice.

Representative (FIG. 3A) full scan chromatogram and (FIG. 3B) mass spectrum of a tumor-bearing mouse. (FIG. 3C) Extraction of the most abundant peak illustrates a 2.5-fold increase in butyrolactone in tumor-bearing versus control mice.

FIGS. 4A-E. Individual ROC curves for individual biomarkers. ROC curve for predicting (no) cancer using (FIG. 4A) 1H-imidazole-4-carboxaldehyde, (FIG. 4B) nahtho[2,3-c]furan-1(3H)-one, 6-hydroxy-5,7-dimethoxy, (FIG. 4C) 2-ethenyl-3-ethylpyrazine, (FIG. 4D) 2,2,6-trimethyl octane, (FIG. 4E) [1,4′]bipiperidinyl-4′-carboxamide, 1-(4′ chlorobenezes) as a biomarker.

FIG. 5. CART diagram for predicting cancer. Cancer was best predicted by 1H-imidazole-4-carboxaldehyde and 2-ethenyl-3-ethylpyrazine.

FIG. 6. ROC curve for the predictive model for ovarian cancer. ROC curve for predicting cancer using

$$log \frac{π}{1 - π} = 1.752 - 0.042 \times 1 H - imidazole - 4 - carboxaldehyde - 0.018 \times 2 - ethenyl - 3 - ethylpyrazine$$

FIG. 7: A SPME portable field breath sampler with mouthpiece is shown.

FIGS. 8A-C: FIG. 8A, FIG. 8B, A SPME portable field breath sampler with mouthpiece configured to collect exhaled breath condensate is shown. FIG. 8C, Breathing through the SPME portable field breath sampler is shown.

### DESCRIPTION OF ILLUSTRATIVE EMBODIMENTS

The present invention is based in part on the discovery that increased or decreased levels of certain VOCs in the breath of a subject can indicate the presence of, or an increased risk of, a cancer in a subject, such as a human patient. In particular aspects, a VOC profile for a patient with a cancer, such as an ovarian or endometrial cancer, is provided. Individual VOC biomarkers that are associated with the presence of a cancer, such as an ovarian or endometrial cancer, are also provided. Breath analysis may be used as a painless, noninvasive technique for separating, detecting the presence or absence of, measuring and/or identifying VOCs associated with a malignancy. In certain embodiments, gas chromatography mass spectroscopy (GC/MS) may be used to detect one or more VOCs from the breath of a subject suspected of having a cancer.

Endogenous volatile organic compounds (VOCs) include blood borne hydrocarbons, oxygen-, sulfur-, and nitrogen-containing compounds and carbon disulfide at ppbv and pptv concentrations. When detected in human breath, VOCs are typically relatively stable and can provide useful insights into different biochemical processes discriminating healthy from disease individuals. Gas chromatography can separate VOCs at ppbv-pptv concentrations which may then be identified with mass spectroscopy (Buszewski et al., 2007). The mechanism by which precise VOCs are generated in the tumor and in the tumor microenvironment is presently not well understood. To the knowledge of the inventors, no correlations between exhaled hydrocarbons or exhaled VOCs and the presence of an ovarian or endometrial carcinoma have been previously identified.

### I. METHODS FOR DETECTING CANCER

The presence or increased risk of a cancer may be detected in a subject via the detecting or measuring one or more VOCs from the breath of a subject. For example, the cancer may be an ovarian cancer such as, e.g., an ovarian epithelial cancer, a colon cancer or colorectal cancer, a pancreatic cancer, a leukemia, or an endometrial cancer. In certain embodiments, the cancer is not a lung cancer or a breast cancer. The endometrial cancer may be a uterine cancer, a cancer from the endometrium, a cervical cancer, a sarcoma of the myometrium, or a trophoblastic disease. The cancer may be metastatic or non-metastatic.

Various types of ovarian cancers may be detected by alterations in one or more VOCs from the breath of a subject. For example, the ovarian cancer may be an epithelial ovarian cancer, a germ cell ovarian cancer, a germ cell ovarian cancer, or a sex cord stromal cancer. The ovarian cancer may be metastatic or non metastatic. Epithelial ovarian tumors are typically derived from the cells on the surface of the ovary. Epithelial ovarian cancer is the most common form of ovarian cancer and occurs primarily in adults. Germ cell ovarian tumors are typically derived from the egg producing cells within the body of the ovary. Germ cell ovarian cancer occurs primarily in children and teens and is rare by comparison to epithelial ovarian tumors. Sex cord stromal ovarian tumors are also rare in comparison to epithelial tumors, and these tumors often produce steroid hormones.

It is anticipated that gas chromatography with or without mass spectroscopy may be used to measure the level or amount of one or more VOC from the breath of a subject. For example, the retention time of OMP, HE, or butyrolactone in GC may be used to detect the presence of or an increased risk of a cancer in a subject.

A. Volatile Organic Compounds

As shown in the below examples, differential levels or amounts of 1H-imidazole-4-carboxaldehyde, nahtho[2,3-c]furan-1(3H)-one,6-hydroxy-5,7-dimethoxy, 2-ethenyl-3-ethylpyrazine, 2,2,6-trimethyl octane, {[1,4′]bipiperidinyl-4′-carboxamide, 1-(4′ chlorobenezes)}, oxime-methoxy-phenyl (OMP), 1-hexano1-2-ethyl (HE), and/or butyrolactone from the breath of a subject can indicate the presence of, or an increased risk of, a cancer. For example, differential or altered levels of one or more of 1H-imidazole-4-carboxaldehyde, nahtho[2,3-c]furan-1(3H)-one,6-hydroxy-5,7-dimethoxy, 2-ethenyl-3-ethylpyrazine, and/or 2,2,6-trimethyl octane in the breath of a subject (e.g., a human patient), in comparison to a control sample or level from a healthy subject, can indicate the presence of or an increased risk of a cancer (e.g., an ovarian cancer) in the subject. The structures of various VOCs that may be detected or measured in certain embodiments or the present invention are shown below.

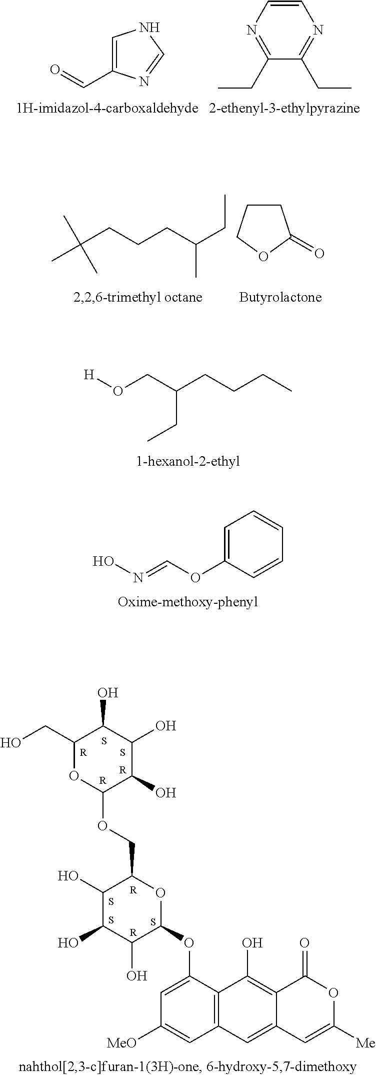

Increased levels of in butyrolacetone, oxime-methoxy-phenyl, and phenol and 1-hexano1-2-ethyl from the breath of a subject can indicate the presence of or an increased risk of a cancer or malignancy in the subject. In certain embodiments, the absence of [1,4′]bipiperidinyl-4′-carboxamide, 1-(4′ chlorobenezes) from the breath of a subject can indicate an increased risk of or the presence of a malignancy. As observed in the below examples, all patients with malignancy displayed an absence of [1,4′bipiperidinyl-4′-carboxamide, 1-(4′ chlorobenezes) in VOCs from breath. With the creation of the following logistic regression equation:

$$ln ( \frac{π}{1 - π} ) = η = 1.752 - 0.042 \times ( 1 H - imidazole - 4 - carboxaldehyde ) - 0.018 \times ( 2 - ethenyl - 3 - ethylpyrazine ) ,$$

both 1H-imidazole-4-carboxaldehyde and 2-ethenyl-3-ethylpyrazine from the breath of a subject can be predictive of malignancy.

Aspects of the present invention are based on the discovery that differences in the VOC profiles from the breath of a subject are different between patients with a cancer, such as an ovarian or endometrial cancer, and patients who are healthy or have only a benign tumor. It is anticipated that differential expression (e.g., increases in, decreases in, or the absence of) other VOCs in the breath of a subject may indicate the presence or absence of a cancer in the subject. As shown in the below examples, the level or intensity of 1H-imidazole-4-carboxaldehyde was observed to be decreased in patients with malignancy. The level or intensity of nahtho[2,3-c]furan-1(3H)-one, 6-hydroxy-5,7-dimethoxy was observed to be increased in patients with malignancy. The level or intensity of 2-ethenyl-3-ethylpyrazine was observed to be decreased in patients with malignancy. The level or intensity of 2,2,6-trimethyl octane was observed to be decreased in patients with malignancy, and [1,4′]bipiperidinyl-4′-carboxamide, 1-(4′ chlorobenezes) was observed to be absent in patients with malignancy.

As shown in the below examples, differential levels or amounts of oxime-methoxy-phenyl (OMP), 1-hexano1-2-ethyl (HE), or butyrolactone can correlate with the presence of a cancer. For example, decreased levels of OMP and/or HE in the breath of a subject, such as a human subject, in comparison to a control sample or level from a healthy subject, can indicate the presence or an increased risk of a cancer, such as an ovarian cancer, in the subject. Increased levels of butyrolactone in the breath of a subject, such as a human subject, in comparison to a control sample or level from a healthy subject, can indicate the presence or an increased risk of a cancer, such as an ovarian cancer.

Preclinical breath samples may be pre-concentrated on a solid phase microextraction (SPME) fiber, thermally desorbed with GC, and volatile organics in the breath can be identified, e.g., with MS. Based on the preclinical findings, a clinical study detected statistically significant differences between patients with and without pathologically confirmed ovarian carcinoma using the breath-based bioassay. Exhaled breath may be collected from patients with pelvic masses, prospectively prior to any treatment or surgical intervention. The area under a ROC curve (AURC) was calculated using AUC as a predictor variable and cancer as the gold standard. ROC curves with AURC >0.7 were selected for further examination. A logistic regression equation using those biomarkers with an AURC >0.7 was created to determine if combining identified markers could improve the ability to distinguish malignancy from benign disease.

As shown in the below examples, an orthotopic preclinical model was used, and breath was collected when animals had palpable tumor. Comparisons of full scan chromatograms of tumor- and non-tumor bearing mice revealed a differentially expressed peak that was identified as butyrolactone (on average 2.5-fold higher in abundance among tumor-bearing mice). There was reproducibility of chromatograms between patients with an average 2-fold higher abdundance of oxime-methoxy-phenyl, phenol and 1-hexanol-2-ethyl among patients with gynecologic malignancy compared to patients with benign disease. Breath samples were collected from 59 patients with pelvic masses: 38 patients with benign disease and 21 patients with epithelial ovarian cancer. Among 1,655 identifiable compounds in the breath, four VOC markers (i.e., 1H-imidazole-4-carboxaldehyde, nahtho[2,3-c]furan-1(3H)-one,6-hydroxy-5,7-dimethoxy, and 2-ethenyl-3-ethylpyrazine, 2,2,6-trimethyl octane) had an AURC >0.7 with one compound being able to distinguish malignancy from benign disease by itself with a sensitivity and specificity of 76% [95% CI=53%-92% ] an d 79% [95% CI=63-90% ], respectively. The four identified markers with an AURC >0.7 were then combined using a logistic regression model. Together, these compounds were able to discrim inate ovarian cancer wit h 86% [95% CI=64-97% ] sensitivity and 79% [95% CI=60-89% ] specificity, respectively. All patients were able to complete breath collection with no identifiable side effects.

B. Gas Chromatography/Mass Spectrometry (GC/MS)

One or more VOC from the breath of a subject may be detected and/or measured via gas chromatography/mass spectroscopy (GC/MS). In certain embodiments, VOCs are collected from the breath of a subject on an solid phase microextraction (SPME) fiber, and the SPME fiber is analyzed using GC/MS, e.g., by thermally desorbing the SPME fiber within a GC inlet and detecting volatile organic peaks in the breath with MS using a NIST library. Thermal desorption may be performed at the GC inle a temperature of, e.g., about 200-350° C. In some embodiments, the SPME fiber is thermally desorbed in the gas chromatography injection port at about 250° C.

In all chromatography, separation occurs when the sample mixture is introduced (injected) into a mobile phase. Gas chromatography (GC), typically uses an inert gas such as helium as the mobile phase. GC/MS allows for the separation, identification and/o quantification of individual components from a biological sample. Various GC/MS tools are commercially available, such as, e.g., a Clams GC/Mass Spectrometer (PerkinElmer, Waltham, Mass., USA), Hewlett Packard 6890 gas chromatograph (Hewlett Packard, Avondale, Pa.), and an Aglient 6890N gas chromatograph coupled with an Agilent 5973 Mass Selective Detector. GC/MS methods which may be used with the present invention include electrospray ionization, matrix-assisted laser desorption/ionization (MALDI), glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). In some embodiments, a triple quadrupole mass spectrometer may be used.

Matrix assisted laser desorbtion ionization time-of-flight mass spectrometry (MALDI-TOF-MS) is an example of a mass spectroscopy method which may be used to measure one or more VOCs from the breath of a subject. Since its inception and commercial availability, the versatility of MALDI-TOF-MS has been demonstrated convincingly by its extensive use for qualitative analysis.

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis.

MALDI-TOF-MS has been used for many applications, and many factors are important for achieving optimal experimental results (Xu et al., 2003). Many studies have focused on the quantification of low mass analytes, such as alkaloids or active ingredients in agricultural or food products (Wang et al., 1999; Jiang et al., 2000; Wang et al., 2000; Yang et al., 2000; Wittmann et al., 2001). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) typically produce improved results. Delayed extraction has also improved the resolution available on modern commercial instruments (Bahr et al., 1997; Takach et al., 1997).

It is anticipated that one or more other analytical approach may be used to measure VOCs from the breath of a subject. In some embodiments, it may be feasible to use a liquid chromatography-tandem mass spectrometry (LC/MS or LC-MS) or ion mobility spectrometry/mass spectrometry (IMS/MS or IMMS) assay to measure a VOCs from the breath of a subject.

C. Statistical Analysis of VOCs

Various statistical methods may be used to identify an increase or a decrease in one or more VOCs relative to control sample(s) or subject(s). For example, methods described by Pepe may be used to select VOCs that are differentially expressed between patients with ovarian cancer and healthy controls from microarray data (Pepe et al., 2003). The first step in this process is to calculate ROC(t₀)=Pr[Y_{VOC}^{D}≧y^{C}(1−t₀)] and

$$pAUC ( {t}_{0} ) = {\int }_{0}^{{t}_{0}} ROC ( t ) d t$$

where y is the value of expression of the VOC, D indicates the cancer group, C indicates the control group, t₀ is some pre-specified false positive rate and y^{C}(1−t₀) is the quantile in the upper tail of the normative range corresponding to t₀. The above statistics, particularly the pAUC (partial area under the curve), can gives an improved indication of separation than traditional measures of discrimination, such as a t-test. In some embodiments, one may choose t₀=10%, which corresponds to the false positive rate found in studies to date when using VOCs to screen for breast cancer. The ROC(t₀) and pAUC(t₀) statistics can be calculated for each VOC, and the VOCs can be ranked according to these statistics. 30 VOCs may be chosen for further evaluation based upon their rankings and evaluate them for stability of selection, which is the probability that the rank of a selected VOC is truly within the selection boundary. For example, Pr[VOC ranked in the top 30]=Pr[Rank(VOC)≦30].

The panel of VOCs may be further narrowed based upon an examination of VOC rank vs. selection probability as well as its ability to discriminate between cancer and non-cancer patients, which will be assessed by graphing ROC(t)=Pr[Y^{D}>u] vs. t=Pr[Y^{C}<u]. This graph may be an ROC curve and can be used to select VOCs with optimal discrimination ability.

After a panel of VOCs has been selected, one can create histograms and summary statistics for this panel by cancer diagnosis. A univariate analysis of this panel may be completed to determine whether the VOCs individually yield any optimal cutpoints that would allow for a reasonable sensitivity and false positive rate.

One may then construct a logistic regression equation using this panel with the ultimate goal being to construct a score w=w(x) based on these VOCs, such that thresholding w would define the desired screening test for ovarian cancer patients. Let d=0 or 1 denote an indicator for ovarian cancer. The inventors will define d by thresholding w, say, d=I(v>c). Prior to calculating w, one may investigate the need for interaction or non-linear terms in the logistic regression model by fitting a CART model; inspection of the fitted regression tree may allow for the identification of interactions or non-linear effects. A logistic regression model for predicting ovarian cancer may be fit using a panel of VOCs and any interaction or non-linear terms as found using the CART model. The maximum likelihood estimates of the logistic regression coefficients can define the desired score w. After determining the optimal cutpoint, 95% confidence intervals may be created for the calculated sensitivity and specificity.

Simulations may be used to estimate Pr[VOC ranked in top 30|VOC is informative] for 30 ovarian cancer patients and 30 healthy controls. Data was simulated for 500 VOCs, of which 470 were created to be non-informative. Specifically, they were equally distributed for both ovarian cancer patients and healthy controls. For the remaining VOCs, values for cancer patients were simulated from a normal distribution with mean 1 and standard deviation 2. Values for the healthy controls were simulated from a standard normal distribution. Therefore, the area under the ROC curve was Φ{(1−0)/(2²+1²)^{1/2}}=0.67 (Reiser and Guttman, 1986). All VOCs were simulated to be independent of each other. In this case, the probability that a particular VOC was ranked in the top 30 given it was an informative VOC was found to be 76.5%.

### II. APPARATUSES FOR THE COLLECTION OF VOLATILE ORGANIC COMPOUNDS FROM BREATH

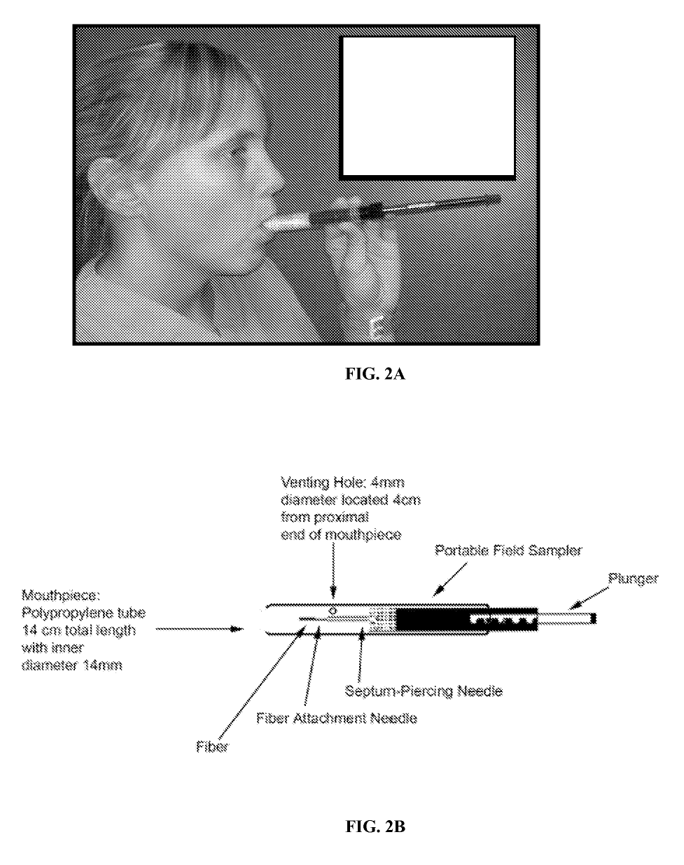

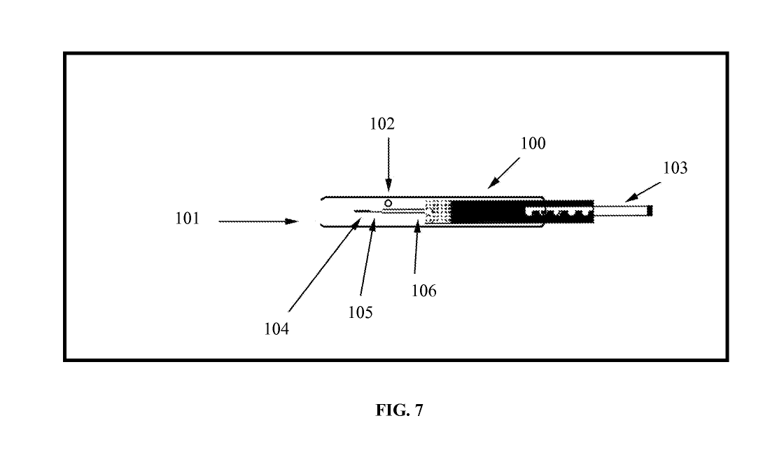

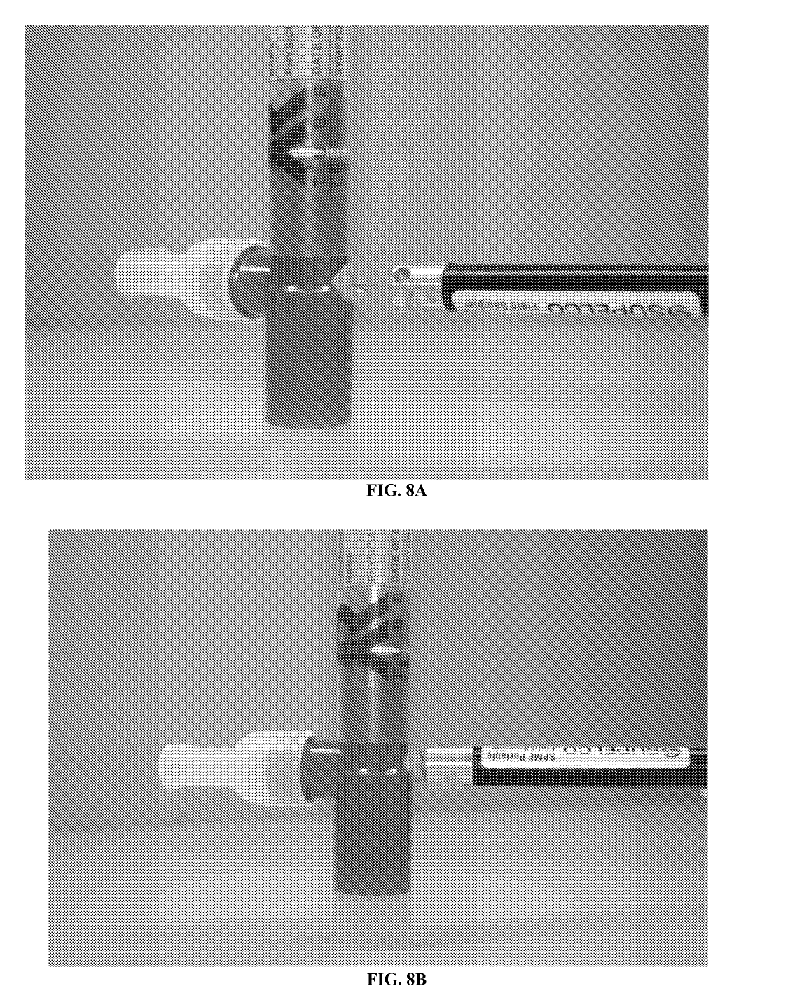

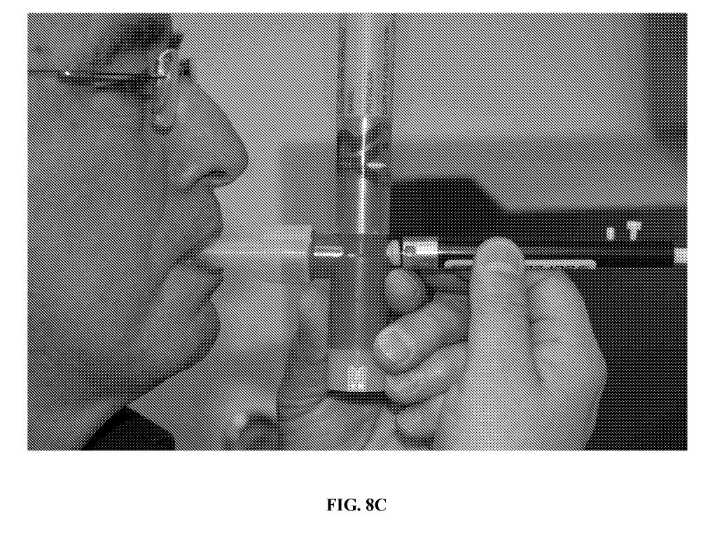

In certain embodiments, the apparatus is a portable apparatus that may be used at a clinic or other point of care location for the collection of volatile organic compounds from breath that may be later chemically analyzed. For example, a SPME portable field sampler with a mouthpiece, e.g., as shown in FIG. 7, FIGS. 2A-B, or FIG. 8 may be used for the collection of one or more volatile organic compound from the breath of a subject. In various embodiments, a subject, such as a human patient, may breathe through a SPME portable field sampler for a period of time (e.g., about 1, 2, 3, 4, 5, 6, 7, 8, 9, 10, 11, 12, 13, 14, or 15 or more minutes), and the SPME fiber may be subsequently analyzed to determine the presence or absence of one or more volatile organic compounds to detect the presence or absence of a cancer in the patient.

A SPME portable field breath sampler with mouthpiece is shown in FIG. 7. The apparatus comprises housing (100) coupled to mouthpiece (101). The mouthpiece may comprise one or more venting hole (102). The venting hole may allow a subject, such as a human subject, to breathe through the mouthpiece (101) while the mouthpiece is in the mouth of the subject. The mouthpiece may be a polymeric tube, such as a polypropylene tube. In certain embodiments, the mouthpiece is about 5-25, about 10-20 cm, or about 5, 6, 7, 8, 9, 10, 11, 12, 13, 14, 15, 16, 17, 18, 19, or 20 cm in length. In some embodiments, the mouthpiece may have an inner diameter of about 10, 11, 12, 13, 14, 15, 16, 17, 18, 19, or 20 mm. In various embodiments, the mouthpiece is a polypropylene tube about 14 cm total length with an inner diameter of about 14 mm. The one or more venting holes may be about 1, 2, 3, 4, 5, 6, 7, or 8 mm in diameter. The one or more venting holes may be about 1, 2, 3, 4, 5, or 6 cm from the proximal end of the mouthpiece. The mouthpiece may be unitary with the housing. Alternately, the mouthpiece may be modular with the housing. The apparatus may comprise a plunger (103) coupled to a fiber attachment needle (105) such that movement of the plunger may move the fiber attachment needle into or out from inside a septum-piercing needle (106). The fiber attachment needle (105) may be coupled to a fiber (104). The fiber may be a SPME fiber, e.g., as further described herein.

An additional configuration of a SPME portable field breath sampler with mouthpiece configured to collect exhaled breath condensate is shown in FIGS. 8A-C. The RTube from Respiratory Research was modified to permit collection of SPME sample simultaneous with exhaled breath condensate. The device may be modified by drilling a 7 mm hole directly opposite the mouthpiece and inserting a 7 mm serum cap. The solid phase microextraction (SPME) device is inserted through the serum cap and the fiber then extended. The patient or volunteer breaths normally through the mouth piece for the specified time. The SPME device is supported by the volunteer's hand as they hold the device. The modified device may be further modified adding an open holder for the SPME to maintain alignment of the fiber in the device. This addition will permit a volunteer to support both SPME and RTube with one hand.

These modifications allow for the collection of exhaled breath condensate (EBC) and volatile organic carbons (VOCs) at the same time. The RT device has a unidirectional device incorporated in its design which permits the volunteer to breath normally through the mouth piece without discomfort or additional effort. The volunteer does not need to remove their mouth from the mouth piece during sample collection. The extended SPME fiber is typically oriented directly in the air path in a optimal location to collect VOCs exhaled with minimum influence from room air influences (i.e., fans, AC outlets, additional breath compounds from other individuals present during sampling, room odors, etc.).

The fiber may deployed out of the portable field sampler with the mouthpiece in place in a subject's mouth, and the subject may then breathe into the mouthpiece normally for a period of, e.g., about 1, 2, 3, 4, 5, 6, 7, 8, 9, 10, 11, 12, 13, 14, 15, 16, 17, 18, 19, or 20 or more minutes. In some embodiments, the patient may breathe into the mouthpiece normally for about 5 minutes to complete the collection. The subject is preferably a mammal, such as a human patient. The SPME fiber may then be placed in the inlet port of a gas chromatography and thermally desorbed generating a chromatograph of volatile organic compounds which are then analyzed by mass spectroscopy.

A. Solid Phase Microextraction (SPME) Apparatus

An apparatus for solid phase microextraction (SPME) may be used to collect one or more volatile organic compounds from the breath of a subject. Solid phase microextraction (SPME) typically uses a relatively quick, solvent-free and field compatible sample preparation method. SPME has been applied to a range of applications including environmental, industrial hygiene, process monitoring, clinical, forensic, food and drug analysis. In SPME, coated fibers are used to isolate and concentrate analytes into a range of coating materials. After extraction, the fibers are transferred, typically with the help of the syringe-like handling device, to an analytical instrument for separation and quantification of the target analytes. The volatile organic compounds, as disclosed herein, may be separated and analyzed in various embodiments via gas chromatography/mass spectrometry (GC/MS).

SPME typically utilizes an extracting phase that is attached to rods made out of various materials. The extracting phase may be a polymeric organic phase that is attached or cross-linked to the rod. In one configuration, the rod may include an optical fiber made of fused silica, which is chemically inert. A polymer layer may be used to protect the fiber against breakage, such as poly(dimethylsiloxane) or polyacrylate. Poly(dimethylsiloxane) can behave as a liquid, which can result in a more rapid extraction compared to polyacrylate, which is a solid. In various embodiments, the silica rods may have a diameter of about 100-200 micrometers and a film thickness ranging from about 10-100 microns. When a coated fiber is placed into an aqueous matrix, the analyte can be transferred from the matrix into the coating. The extraction is typically considered to be complete when the analyte has reached an equilibrium distribution between the matrix and fiber coating.

SPME fibers are typically rather fragile; thus, a SPME fiber may be included in a syringe or micro-syringe device. Movement of a syringe plunger can allow a SPME fiber to be extruded from the needle for extraction or introduction into an analytical instrument. By moving the plunger up, the fiber is protected in the needle during both storage and penetration of injection-port septa. An example of a SPME portable field sampler with a mouthpiece is shown in FIG. 7, FIGS. 2A-B, and FIGS. 8A-C and may be used for the collection of one or more volatile organic compound from the breath of a subject. The plunger may be coupled or attached to the SPME fiber such that movement of the plunger may be protected or extruded from a needle. The SPME fiber may be attached to a fiber attachment needle, which may be retractable to or from a septum piercing needle.

A SPME method for semivolatile analysis may involve inserting the fiber device into an aqueous sample matrix, pushing the plunger to expose the fiber, retracting the fiber into the needle when equilibrium has been reached, and finally introducing the fiber into an analytical instrument, such as, e.g., a GC/MS instrument. During desorbtion of the analyte, the polymeric phase is typically cleaned and therefore ready for reuse. The absence of solvent in SPME can, in various embodiments, increase the speed of separation, increase throughput, and/or allow for the use of simpler instruments.

B. Solid Phase Microextraction (SPME) Fibers

An apparatus for the collection of one or more VOCs from breath may comprise a solid phase microextraction fiber. A variety of SPME fibers may be used for collection of one or more volatile organic compound from the breath of a subject. For example, the SPME fiber may comprise a carboxen and polymethylsiloxane (CAR/PDMS) coating, a divinylbenzene/carboxen/polydimethylsiloxane (DVB/CAR/PDMS) coating, a polydimethylsiloxane (PDMS) metal alloy, Carbopack-Z fibers, polyacrylate (PA), a Carbowax-polyethylene glycol (PEG) coating, a Carbowax/template resin (CW/TPR) coating, or a a polydimethylsiloxane/divinylbenzene (PDMS/DVB) coating. A flexible metal alloy may be used in the needle, plunger, and fiber core. A needle may be attached to the SPME fiber, e.g., a 23 or 24 gauge needle.

Additional SPME fibers and methods are known in the art and may be used with the present invention (e.g., see Mitra and Somenath, 2003; Pawliszyn, 2009; Pawliszyn, 1997; and Pawliszyn, 1999, which are incorporated herein by reference in their entirety).

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Measurement of VOCs in Exhaled Breath of Nude Mice

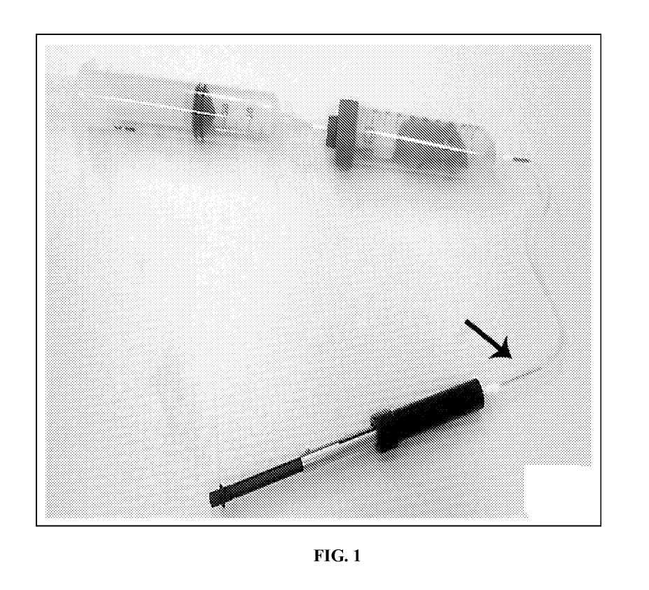

To identify the feasibility of detecting differences in the breath of ovarian cancer patients, a previously described orthotopic mouse model of ovarian carcinoma was utilized. Female athymic nude mice were purchased from the National Cancer Institute-Frederick Cancer Research and Development Center (Frederick, Md.) and housed in specific pathogen-free conditions. Animals were cared for in accordance with the guidelines set forth by the American Association for Accreditation for Laboratory Animal care and the U.S. Public Health Service Policy on Human Care and Use of Laboratory Animals. All studies were approved and supervised by the University of Texas M.D. Anderson Cancer Center Institutional Animal Care and Use Committee. The human ovarian cancer cell line, HeyA8, was grown in culture and incubated with EDTA, centrifuged, washed twice with Hank's balanced salt solution and resuspended at a concentration of 1.25×10⁶ cells/mL. Each mouse was injected intraperitoneally with 200 μA of cell suspension. Once tumors were palpable by physical examination, breath samples were collected using the collection chamber and pre-concentrated on a SPME depicted in FIG. 1. Mice were housed in the collection chamber for a total of 6 minutes. The air was refreshed in 60 cc aliquots every 90 seconds. After the pre-concentration process, the SPME fiber in the manual holder was thermally desorbed in the gas chromatography injection port at 250° C. for 10 seconds with the splitless injection mode. The GC/MS analysis was performed using an Aglient 6890N gas chromatograph coupled with an Agilent 5973 Mass Selective Detector. The VOCs were separated on an Agilent DB-FFAP column (30 m×0.25 mm, 0.25 m film thickness). The temperature gradient was set for 40° C. for 5 minutes, then 10° C. per minute to 250° C. and finally at 250° C. for 4 minutes. The total run time was 30 minutes. Each SPME fiber was baked in the GC inlet at 250° C. for 30 seconds after sample injection.

Measurement of VOCs in Exhaled Breath of Patients with Benign Disease versus Malignancy

Subjects

The Institutional Review Board of the University of Texas M.D. Anderson Cancer Center approved the conduct of this research study. All subjects gave their signed informed consent to participate. Candidates for the epithelial ovarian cancer cohort were recruited from patients referred to the Department of Gynecologic Oncology at the University of Texas M.D. Anderson Cancer Center with suspected advanced epithelial ovarian cancer prior to therapeutic intervention. Patients with a history of treated epithelial ovarian cancer who had received chemotherapy within 6 months of study entry were excluded. All patients underwent either surgical resection of their primary malignancy or percutaneous biopsy to obtain a tissue diagnosis prior to administration of combination taxane and platinum chemotherapy. Admission to the epithelial ovarian cancer group was based on the reported histopathology of the patient's surgical or biopsy specimens. The pathologic stage of disease was determined according to the International Federation of Gynecology and Obstetrics staging system for ovarian cancer by examination of the pathological tumor specimen. Candidates for the control cohort were recruited from patients referred to the Department of Gynecologic Oncology at the University of Texas M.D. Anderson Cancer Center with suspected benign disease prior to therapeutic intervention. Subjects were entered into the control group based on the reported histopathology of benign disease or ovarian tumors of low malignant potential after review of patient's surgical specimens. Pathologists without knowledge of the breath test results interpreted tissue samples. Analyses of breath VOCs were performed by EF without knowledge of the pathologic findings. Breath collection was performed by asking subjects to breathe normally through the disposable mouthpiece of a portable breath collection apparatus for 5 minutes (FIG. 2A and FIG. 2B). VOCs were pre-concentrated on a solid phase microextraction (SPME) fiber composed of polydimethylsiloxane (PDMS) and carboxen, thermally desorbed with gas chromatography and identified with mass spectroscopy.

Carboxen-PDMS sampling devices were purchased from Supelco, Inc. Each SPME device was conditioned prior to using. To condition the SPME devices, the fiber protective needle was extended through the septum plug and inserted into the inlet of the Agilent 6890 GC instrument. The carboxen-PDMS inside the needled was then deployed into the inlet set at 280° C. for 3 minutes. After conditioning, the fiber was retracted into the protective needle and the needle was removed from the GC inlet. The needle was then completely retracted behind the septum plug and stored at 5° C. to protect the conditioned carboxen-PDMS filter from ambient air exposure until used for patient sample collection.

Following collection of patient breath samples the SPME devices were stored at 5° C. SPME samples were analyzed by direct injection into the inlet of the GC as soon after collection as possible to minimize any loss of VOCs. Patient SPME breath samples were analyzed by manual injection into an Aglient 6890/5973 GC-MSD. As in the initial conditioning, the SPME needle protecting the fiber was inserted into the GC inlet set at 280° C. and the fiber was then deployed. The breath samples were injected into the GC column for 30 seconds using splitless mode. The SPME fiber was held in the inlet for a total of 2 minutes to complete desorption of all captured VOCs. After two minutes the SPME fiber was withdrawn from the GC inlet and stored at 5° C. until they were reconditioned for additional use.

Patient sample data was acquired using Agilent ChemStation software. The ChemStation files were converted to AIA format (aka: ANDI/netCDF mass spectrometry data interchange format) and exported into Water's MassLynx software. The data files were then converted into Water's*.raw format and analyzed using Water's MassLynx software to screen for markers that differentiate benign versus malignant patient samples. Markers were defined on the basis of their retention time and m/z (mass to charge ratio). Selected markers were deconvoluted using Water's ChromaLynx software and putatively identified by comparison to the NIST11/2011/EPA/NIH mass spectral library (National Institute of Standards and Technology).

Statistical Methods

For the clinical samples, regardless of the fold-change or statistical significance, receiver operating characteristic (ROC) curves were calculated for candidate biomarker in the test cohort of patients. The area under the ROC curve (AURC) using AUC as the predictor variable and cancer (vs. benign, yes/no) as the gold standard was calculated. Those ROC curves with AURC >0.7 were selected for further examination using Cartesian and Regression Tree (CART) analysis to determine which of these biomarkers were most influential in predicting cancer and whether any interactions between biomarkers occurred. The results of the CART analysis was then used to create a logistic regression equation to predict cancer. Each individual's predicted logits

$$( {η}_{i} = ln [ \frac{{π}_{i}}{1 - {π}_{i}} ] = {\hat{β}}_{0} + {\hat{β}}_{1} {X}_{1 i} + {\hat{β}}_{2} {X}_{2 i} + … )$$

were calculated and also examined for their ability to distinguish malignant tumors using an ROC curve.

### Example 2

### A Breath-Based Bioassay for Ovarian Cancer

Comparisons of VOCs in Tumor Bearing versus Control Mice

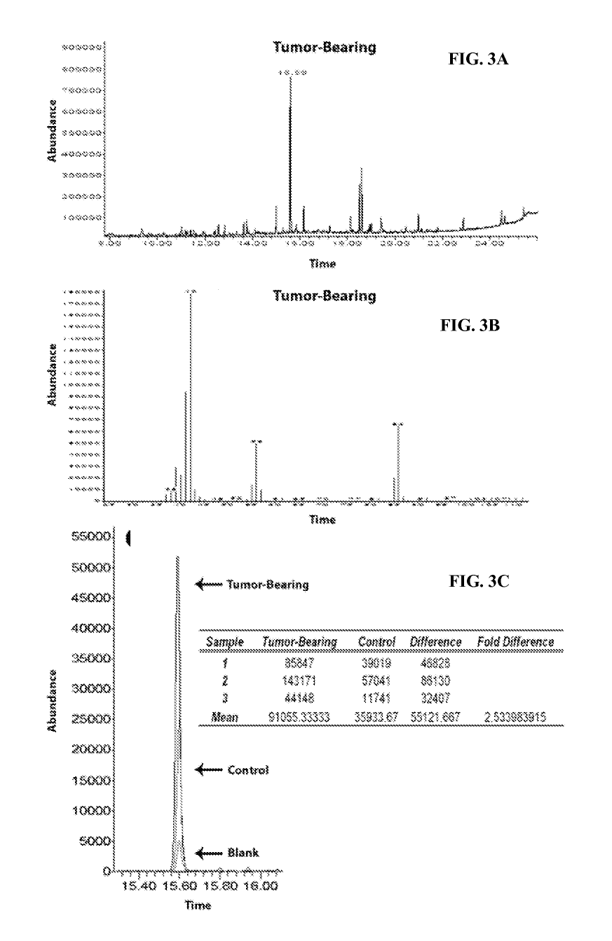

Comparisons of full scan chromatograms of tumor-bearing and control mice revealed a peak that was more intense for the tumor-bearing samples. A full scan chromatogram of the peaks from a tumor-bearing mouse is shown in FIG. 3A. The retention time peak was 15.60 minutes and was reproducible within 0.01 minutes. The mass spectrum of the peak is shown in FIG. 3B. It was identified as butyrolactone with a library search match quality score of 91. The library search match quality score represents the probability that the unknown is correctly identified as the reference. Values greater than 90 are considered very good matches. Values less than 50 mean that substantial differences exist between the unknown and the reference. Differences in probability values of +/− are generally not significant. From the full scan chromatogram, the most abundant ion at 86 m/z was extracted for each sample and its abundance was compared in terms of area count. On average 2.5-fold increase in abundance was calculated among the tumor-bearing and non-tumor-bearing mice (FIG. 3C), providing evidence supporting the feasibility of using this technology for discrimination of the presence of ovarian cancer.

Patient Characteristics

No subject reported any adverse effects of donating a breath sample. Characteristics of subjects in the primary ovarian cancer and control groups are shown in Table 1. Patients in the ovarian cancer cohort were significantly older than those with benign disease (60.7 years vs. 52.3 years, p=0.03). Patients were excluded from analysis if another pathology (i.e., metastatic cancer) was demonstrated of if staging information or other demographic data was not available. Exhaled breath was collected from 59 patients with pelvic masses prior to any therapy or surgical intervention. Thirty-eight patients ultimately had benign disease and 21 patients were noted to have epithelial ovarian cancer.

TABLE 1
Patient Characteristics

| | Mean | Range |
|---|---|---|
| Benign Disease | | |
| Age (y) | 52.3 | 18-79 |
| Ca125 | 109.48 | <7-876.4 |
| Malignancy | | |
| Age (y) | 60.7 | 41-80 |
| Ca125 | 1289.3 | 31.2-6415.3 |

Feasibility of Detecting Differences in Human Breath

Interim analysis of breath samples revealed reproducibility of chromatograms between patients and an average 2-fold high abdundance of oxime-methoxy-phenyl, phenol and 1-hexanol-2-ethyl among patients with gynecologic malignancy compared to patients with benign disease.

Prediction of Ovarian Cancer

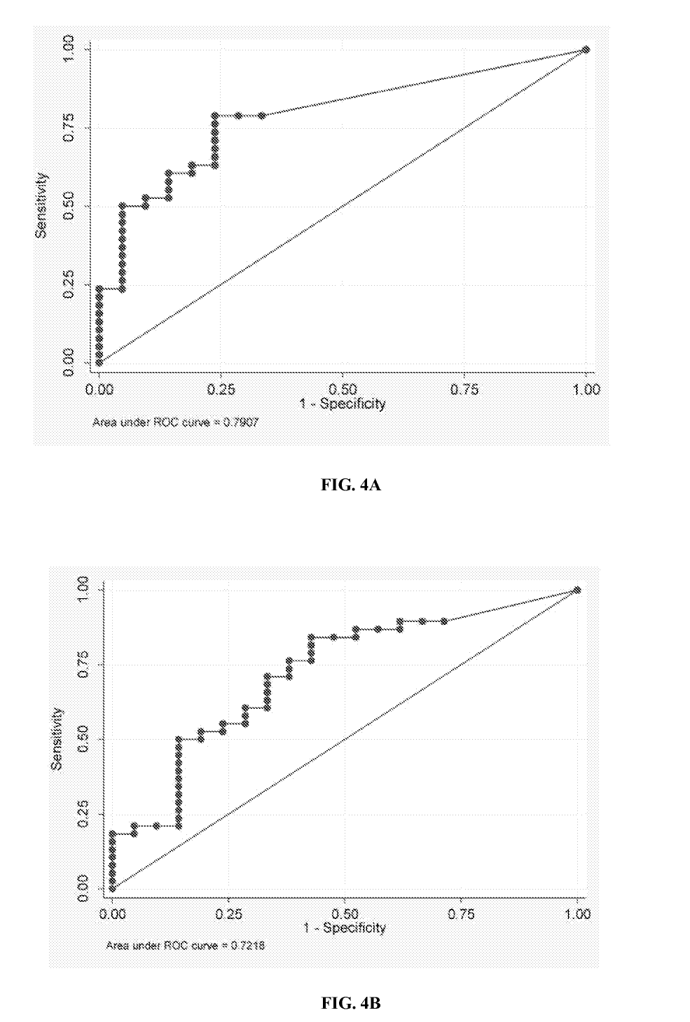

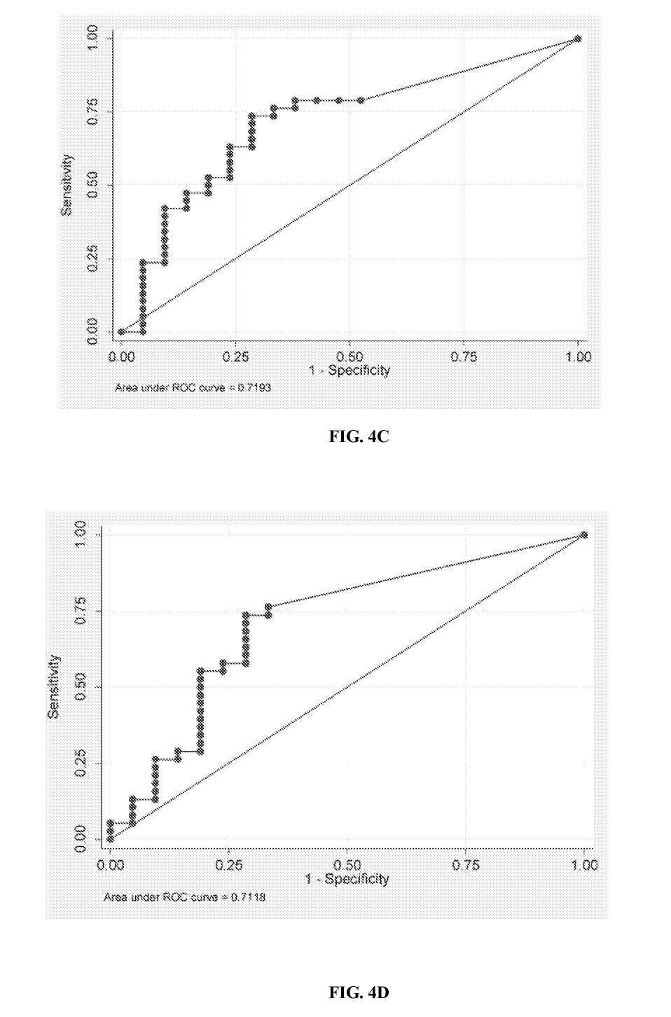

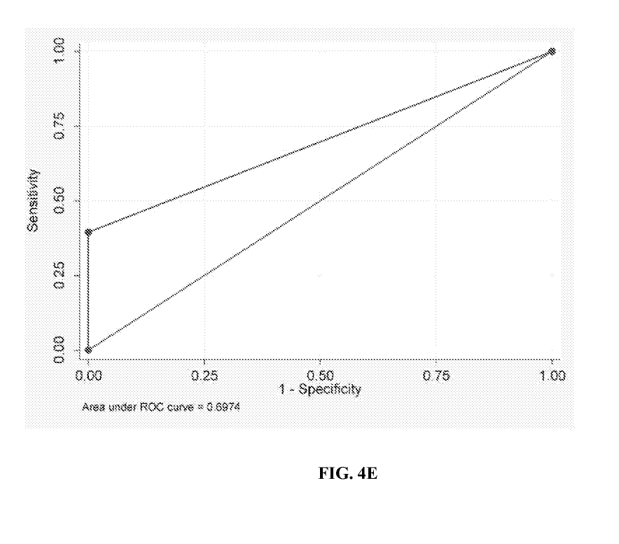

Receiver operating characteristic (ROC) curves displaying the results of the breath test in the training set are shown in FIGS. 4A-E. Among 1,655 identifiable compounds in the breath, four compounds (1H-imidazole-4-carboxaldehyde; nahtho[2,3-c]furan-1(3H)-one, 6-hydroxy-5,7-dimethoxy; 2-ethenyl-3-ethylpyrazine; and 2,2,6-trimethyl octane) had an AURC >0.7 (Table 2) with one compound (1H-imidazole-4-carboxaldehyde) being able to distinguish malignancy from benign disease by itself with a sensitivity of 76% [95% CI=53%-92%] and a specificity of 79% [95% CI=60%-89%] and an area under the ROC curve of 0.79 [95% CI=0.68-90]. One additional compound {[1,4′]bipiperidinyl-4′-carboxamide, 1-(4′ chlorobenezes)} was also selected for further examination because although its AURC was 0.69, all patients with malignant tumors had an AURC of 0.

TABLE 2
VOCs with an AURC >0.7

| Compound | AUC | 95% CI |
|---|---|---|
| 1H-imidazole-4-carboxaldehyde | 0.791 | 9.678-0.903 |
| nahtho[2,3-c]furan-1(3H)-one, 6-hydroxy-5,7- | 0.722 | 0.583-0.861 |
| dimethoxy | | |
| 2-ethenyl-3-ethylpyrazine | 0.719 | 0.582-0.857 |
| 2,2,6-trimethyl octane | 0.712 | 0.571-0.853 |
| [1,4′]bipiperidinyl-4′-carboxamide, 1- | 0.697 | 0.619-0.776 |
| (4′ chlorobenezes) | | |

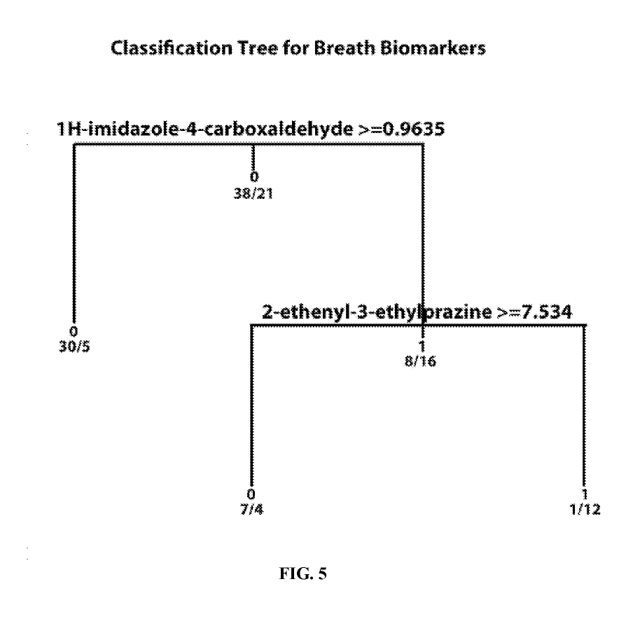

The CART analysis (FIG. 5) indicated that cancer can be best predicted by 1H-imidazole-4-carboxaldehyde and 2-ethenyl-3-ethylpyrazine using the following rule: (1) if the AURC of 1H-imidazole-4-carboxaldehyde >0.9635, (i) then predict no cancer; (ii) otherwise, examine 2-ethenyl-3-ethylpyrazine; (2) if the AURC of 2-ethenyl-3-ethylpyrazine >7.534, (i) then predict no cancer; (ii) otherwise predict cancer. The sensitivity and specificity of this rule is only 57.1% [95% CI=34%-78.2%] but the specificity is 97.4% [95% CI: 86.2%-99.9%]. The sensitivity can be improved by using the AURC of 1H-imidazole-4-carboxaldehyde and 2-ethenyl-3-ethylpyrazine to predict cancer via a logistic regression equation. The resulting equation is:

$$ln ( \frac{π}{1 - π} ) = η = 1.752 - 0.042 \times ( 1 H - imidazole - 4 - carboxaldehyde ) - 0.018 \times ( 2 - ethenyl - 3 - ethylpyrazine )$$

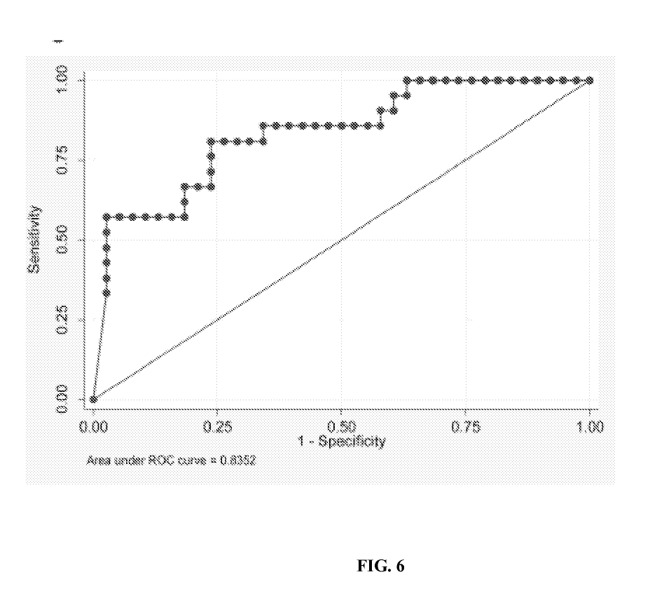

The AURC for this logistic regression equation is 0.835 [95% CI=727-9.44]. The ROC curve using the predicted logits generated from the above equation is displayed in FIG. 6. An examination of various cutpoints indicated that using ρ=>-0.13 to predict cancer yields a sensitivity of 81.0% [95% CI=58.1%-94.6%] and a specificity of 76.3% [95% CI =59.8%-88.6% ].

A unique signature of VOCs was identified as being associated with malignancy among patients with pelvic masses scheduled for the surgical or chemotherapeutic intervention. The key findings of this study are that significant differences were noted between the breath of cancer patients and those without malignancy and the absence of 1H-imidazole-4-carboxaldehyde served as the single best predictor of cancer and the specificity of this marker was improved by sequentially evaluating expression of 2-ethenyl-3-ethylpyrazine in a logistic regression equation. The noninvasive sampling process makes breath collection safe and easy even for nonclinical personnel and modern analytical instruments can be used to detect the VOCs in the breath that are characteristic of epithelial ovarian malignancy.

All of the compositions, methods, and apparatuses disclosed and claimed herein can be made and executed without undue experimentation in light of the present disclosure. While the compositions and methods of this invention have been described in terms of preferred embodiments, it will be apparent to those of skill in the art that variations may be applied to the compositions, methods, and apparatuses and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bahr et al., J. Mass. Spectrom., 32:1111, 1997.
Bast et al., Int. J. Gynecol. Cancer, 15(3):274-281, 2005.
Buszweski et al., Biomed. Chromatogr., 21(6):553-566, 2007.
Duncan et al., Rapid Commun. Mass Spectrom., 7:1090, 1993.
Jiang et al., J. Agric. Food Chem., 48:3305, 2000.
Mitra and Somenath, In: Chapter 2: Principles of Extraction, Sample Preparation Techniques in Analytical Chemistry, Wiley-Interscience, 113, 2003.
Pawliszyn, In: Applications of Solid Phase Microextraction, Royal Society of Chemistry, 1999.
Pawliszyn, In: Handbook of Solid Phase Microextraction, Chemical Industry Press, 2009.
Pawliszyn, In: Solid Phase Microextraction: Theory and Practice, Wiley-VCH, 1997.
Pepe et al., Biometrics, 59(1):133-142, 2003.
Ransohoff, Nat. Rev. Cancer, 4(4):309-314, 2004.
Reiser and Guttman, Technometrics, 28:253-257, 1986.
Takach et al., J. Protein Chem., 16:363, 1997.
Wang et al., J. Agric. Food. Chem., 47:1549, 1999.
Wang et al., J. Agric. Food. Chem., 47:2009, 1999.
Wang et al., J. Agric. Food. Chem., 48:2807, 2000.
Wang et al., J. Agric. Food. Chem., 48:3330, 2000.
Wittmann et al., Biotechnol. Bioeng., 72:642, 2001.
Xu et al., Mass Spectrom Rev., 22(6):429-40, 2003.
Yang et al., J. Agric. Food. Chem., 48:3990, 2000.

1. A method of detecting the presence of, or an increased risk of, an ovarian or endometrial cancer in a subject, comprising detecting or measuring one or more volatile organic compound (VOC) from the breath of the subject; wherein differential expression the VOC as compared to a control indicates that the subject has, or has an increased risk of having, the cancer.

2. The method of claim 1, wherein the one or more VOC comprises at least one of 1H-imidazole-4-carboxaldehyde, nahtho[2,3-c]furan-1(3H)-one,6-hydroxy-5,7-dimethoxy, 2-ethenyl-3-ethylpyrazine, 2,2,6-trimethyl octane, and {[1,4′]bipiperidinyl-4′-carboxamide, 1-(4′ chlorobenezes)}; wherein a decreased level of 1H-imidazole-4-carboxaldehyde, 2-ethenyl-3-ethylpyrazine, 2,2,6-trimethyl octane, or indicates that the subject has, or has an increased risk of having, the cancer; wherein a decreased level of or the absence of {[1,4′]bipiperidinyl-4′-carboxamide, 1-(4′ chlorobenezes)} indicates that the subject has, or has an increased risk of having, the cancer; and wherein an increased level of {nahtho[2,3-c]furan-1(3H)-one, 6-hydroxy-5,7-dimethoxy} indicates that the subject has, or has an increased risk of having, the cancer.

3. The method of claim 2, wherein the one or more VOC comprises at least two of 1H-imidazole-4-carboxaldehyde, nahtho[2,3-c]furan-1(3H)-one,6-hydroxy-5,7-dimethoxy, 2-ethenyl-3-ethylpyrazine, 2,2,6-trimethyl octane, and {[1,4′]bipiperidinyl-4’-carboxamide, 1-(4′ chlorobenezes)}.

4. The method of claim 3, wherein the one or more VOC comprises 1H-imidazole-4-carboxaldehyde and 2-ethenyl-3-ethylpyrazine.

5. The method of claim 3, wherein the one or more VOC comprises at least three of 1H-imidazole-4-carboxaldehyde, nahtho[2,3-c]furan-1(3H)-one,6-hydroxy-5,7-dimethoxy, 2-ethenyl-3-ethylpyrazine, 2,2,6-trimethyl octane, and {[1,4′]bipiperidinyl-4’-carboxamide,1-(4′ chlorobenezes)}.

6. The method of claim 5, wherein the one or more VOC comprises all of 1H-imidazole-4-carboxaldehyde, nahtho[2,3-c]furan-1(3H)-one,6-hydroxy-5,7-dimethoxy, 2-ethenyl-3-ethylpyrazine, and 2,2,6-trimethyl octane.

7. The method of claim 1, wherein the one or more VOC comprises oxime-methoxy-phenyl, 1-hexano1-2-ethyl, or butyrolactone; wherein an increased level of butyrolactone or a decreased level of oxime-methoxy-phenyl or 1-hexano1-2-ethyl, as compared to a control indicates that the subject has, or has an increased risk of having, the cancer.

8. The method of claim 1, wherein the subject is a human.

9. The method of claim 1, wherein the method comprises having the subject breathe onto a solid phase microextraction (SPME) fiber.

10. The method of claim 9, wherein the SPME fiber is comprised in a portable apparatus or a point of care apparatus.

11. The method of claim 10, wherein the VOC is detected from the SPME fiber via gas chromatography/mass spectroscopy (GC/MS).

12. The method of claim 1, wherein said measuring comprises detecting the VOC via gas chromatography (GC).

13. The method of claim 1, wherein said measuring comprises detecting the VOC via gas chromatography/mass spectroscopy (GC/MS).

14. The method of claim 1, wherein the subject has the ovarian or endometrial cancer.

15. The method of claim 1, wherein the method further comprises administering an anti-cancer therapy to the subject.

16. The method of claim 1, wherein the cancer is an ovarian cancer.

17. An apparatus comprising a mouthpiece coupled to a housing, wherein the housing comprises a solid phase microextraction fiber, wherein the apparatus is configured to capture one or more volatile organic compound (VOC) the breath of a subject on the solid phase microextraction fiber when the subject breathes into the mouthpiece.

18. The apparatus of claim 17, wherein the apparatus further comprises an apparatus configured to collect exhaled breath condensate.

19. The apparatus of claim 17, wherein the solid phase microextraction fiber contains one or more of oxime-methoxy-phenyl, 1-hexano1-2-ethyl, and butyrolactone from the breath of the subject.

20. The apparatus of claim 17, wherein the solid phase microextraction fiber comprises a fiber selected from the list consisting of carboxen and polymethylsiloxane (CAR/PDMS), divinylbenzene/carboxen/polydimethylsiloxane (DVB/CAR/PDMS), polydimethylsiloxane (PDMS) metal alloy, Carbopack-Z fiber, polyacrylate (PA), Carbowax-polyethylene glycol (PEG), Carbowax/template resin (CW/TPR), and polydimethylsiloxane/divinylbenzene (PDMS/DVB).

21. The apparatus of claim 20, wherein the solid phase microextraction fiber is a carboxen and polymethylsiloxane (CAR/PDMS) solid phase microextraction fiber.

22. The apparatus of claim 20, wherein the solid phase microextraction fiber is coupled to a needle.

23. The apparatus of claim 17, wherein the apparatus further comprises a septum piercing housing needle coupled to the solid phase microextraction fiber.

24. The apparatus of claim 17, wherein the mouthpiece and the housing are unitary.

25. The apparatus of claim 17, wherein the mouthpiece and the housing are modular.

26. The apparatus of claim 17, wherein the apparatus comprises a plunger, wherein the plunger is coupled to the solid phase microextraction fiber such that movement of the plunger can result in the movement of the solid phase microextraction fiber into or out from the needle.

27. The apparatus of claim 17, wherein the mouthpiece has an internal diameter of about 10 mm to about 20 mm.

28. The apparatus of claim 27, wherein the mouthpiece has an internal diameter of about 14 mm.

29. The apparatus of claim 17, wherein the housing comprises an aperture or venting hole, wherein the aperature or venting hole allows the mammalian subject to breathe through the mouthpiece.

30. The apparatus of claim 29, wherein the housing comprises one aperture or venting hole.

31. The apparatus of claim 30, wherein the aperture or venting hole is about 2-10 mm in diameter.

32. The apparatus of claim 29, wherein the housing comprises more than one aperture or venting hole.

33. The apparatus of claim 29, wherein the aperture or venting hole is about 2-10 cm from the proximal end of the mouth piece.
